(19) United States
(12) Patent Application Publication
(10) Pub. No.: US 2015/0083141 A1
(43) Pub. Date: Mar. 26, 2015

(54) HEARING PROTECTION MEANS FOR USE WITH A MAGNETIC RESONANCE TOMOGRAPHY DEVICE AND MAGNETIC RESONANCE TOMOGRAPHY DEVICE WITH HEARING PROTECTION MEANS

(71) Applicant: SIEMENS AKTIENGESELLSCHAFT, 80333 MUNCHEN (DE)

(72) Inventors: FRIEDERIKE HERTLE, ERLANGEN (DE); JOCHEN RICK, BUCKENHOF (DE)

(21) Appl. No.: 14/489,549

(22) Filed: Sep. 18, 2014

(30) Foreign Application Priority Data
Sep. 25, 2013 (DE) 102013219235.5

(51) Int. Cl. A61F 11/14; G01R 33/28

(52) U.S. Cl. CPC A61F 11/14; G01R 33/28
USPC 128/866

(57) ABSTRACT

A hearing protection means for use with a magnetic resonance tomography device, comprising two noise-protection earpieces for external coverage of two outer ears of an examination object and at least one elastic holder means for affixing the noise-protection earpieces around the outer ears of the examination object, wherein the two noise-protection earpieces comprise a non-magnetic material and have a sound-deadening function is provided. The at least one elastic holder means has a strap-like shape, is able to be disposed on the head of the examination object circumferentially, in this case running along the area of the lower jaw and the outer ear region, and is able to be adapted in its circumferential length such that a predetermined tension is able to be set over the entire circumferential length. The present disclosure further describes a magnetic resonance tomography device which includes a corresponding hearing protection means.

### CROSS-REFERENCE TO RELATED APPLICATIONS

This application claims priority to DE Application No. 102013219235.5 having a filing date of Sep. 25, 2013, the entire contents of which are hereby incorporated by reference.

### FIELD OF TECHNOLOGY

The following relates to a hearing protection means configured for use with a magnetic resonance tomography device. In addition the present disclosure relates to a magnetic resonance tomography device with a hearing protection means.

### BACKGROUND

Magnetic resonance tomography, MRT or MR for short, is an imaging method which may be employed in medical diagnostics for representing structures and functions of tissue and organs in human or animal bodies. Physically it may be based on the principles of nuclear magnetic resonance (NMR), especially field gradient NMR and may also be referred to as nuclear magnetic tomography. Devices with which magnetic resonance tomography can be carried out may be called magnetic resonance tomography devices. A problem which occurs during the operation of a magnetic resonance tomography device may be the generation of noise. The extremely rapid switchover of magnetic fields as a function of the operating principle may cause loud noises to frequently occur during the imaging. Depending on a selected sequence these noises may be perceived as cheeping, knocking, buzzing, clattering or sawing noises. The repetition frequencies of the imaging can extend into the kHz range in such cases. Measurements of an effective sound pressure level with use of an XQ gradient may lie at 88.3 dB(A) for staff in the examination room. The manufacturer of a magnetic resonance tomography device therefore prescribes the use of hearing protection with an SNR (single number rating) of SNR=7 dB or more. For a patient who is mostly moved for image recording into a tunnel of a magnetic resonance tomography device, noise levels of up to 126 dB(A) may occur, which should be attenuated to 99 dB(A). Thus for example suitable hearing protection may offer an SNR=30 dB or more.

With head examinations in particular, the spatial constriction in the head coil through which a better examination result is achieved, promotes the need for a compact hearing protection design. In some instances, small children may present a problem because no hearing protection of a suitable size currently exists which insures noise protection, even during longer examinations which can last up to an hour. The currently known hearing protection presents a danger to the small child due to the risk of the protection means slipping off

Conventional hearing protection means may take the form of headphones for example, which are designed for the examination of adults and thus also for the size of their heads. With these headphones the noise-protection earpieces which cover the ears, are connected by a strap. In practice these are also used in some cases with small children. The disadvantage of these headphones in the examination of babies or small children that the earpieces are too large and do not make a close fit around the ears possible. Furthermore, as a result of the larger design, the pressure is often not sufficient to guarantee a secure hold and noise protection for the duration of an examination period of up to an hour. Often, an additional or alternative hearing protection in the form of an earplug is worn. Another design of a hearing protection means that may be used include sound-deadening earpieces without a holder, may be attached to the ear. These alternatives present a danger of not staying securely affixed to the patient for a measurement period of an hour.

### SUMMARY

An aspect relates to a hearing protection, which by comparison with today's usual hearing protection means, offers a space-saving design and an improved fit on the patient.

This aspect may be achieved with a hearing protection means for use with a magnetic resonance tomography device with the features of the first independent claim and a magnetic resonance tomography device with hearing protection means with the features of the second independent claim. Advantageous embodiments are described in the dependent claims.

A basic idea of the disclosure is a hearing protection means for use with a magnetic resonance tomography device. The hearing protection means comprises two noise-protection earpieces for covering the two outer ears of an examination object and at least one elastic holder means for fixing the noise-protection earpieces around the outer ears of the examination object, wherein the two noise-protection earpieces comprise a non-magnetic material and have a sound-deadening function. The at least one elastic holder means has a strap-like shape, is able to be disposed on the head of the examination object circumferentially, running along the area of the lower jaw and the outer ear region, and is able to be adapted in a circumferential length so that a predetermined tension is able to be set over the entire circumferential length.

The disclosed hearing protection means is suitable for use with a magnetic resonance tomography device. The hearing protection means comprises two noise-protection earpieces for covering the outer ears of an examination object, e.g. of a human patient. They consist of non-magnetic materials and have a sound-deadening function which is achieved for example by suitable and known sound-deadening materials. At least one elastic holder means, e.g. a reversible, flexible strap, serves to fix the noise-protection earpieces around the outer ears of the examination object. The at least one elastic holder means has a strap-like shape and can be arranged around the circumference of the head of the examination object. In a state in which it is disposed on the head of the examination object, the elastic holder means runs in a frontal plane, in the area of the lower jaw and the outer ear region. The elastic holder means can also be referred to as a strap-type holder means or be interpreted as a chin strap. The circumferential length of the elastic holder means is adaptable such that a predetermined tension is able to be set over the entire circumferential length. The degree of tension is generally a compromise between wearing comfort when wearing the hearing protection means, (a lower tension would be desirable) and the holding function to be achieved(a high tension would be desirable here). A compromise value can be found for example by series of tests or predetermined by a manufacturer.

In the exemplary embodiment, the elastic holder means include a hook and loop fastener for adapting the circumferential length.

Hook and loop fasteners are generally releasable fastening means which mostly have a loop component and a mating hook component. Through an elongated version of a hook and loop connection in the form of a hook and loop strap or hook and loop strip, the length the elastic holder means, can be adapted to an individual head size and head shape.

In some embodiments, the two noise-protection earpieces may each comprise a feedthrough means for longitudinal movement of the two earpieces.

The earpieces may not be permanently connected to the elastic holder means. By means of feedthroughs on the distal side of the noise-protection earpieces, i.e. on the side facing away from the head in each case, the elastic holder means may be guided through the noise-protection earpieces such that a caudal/cranial movement of the noise-protection earpieces is possible. This may allow a further adaptation possibility to the head shape and head size of the person being examined. Feedthroughs can be realized for example by brackets which may be disposed on the outer sides of the noise-protection earpieces and form loops with the outer sides, through which the elastic holder means can be guided. Closed feedthroughs or open feedthroughs are conceivable. With an open feedthrough the loop formed by the described bracket is not closed.

The hearing protection means may comprise at least one further elastic holder means. The further elastic holder means may be able to be disposed circumferentially and in parallel to the transversal plane of the examination object and in the area of the head of the examination object.

The further elastic holder means, e.g. a further reversible flexible strap, may supplement the previously described elastic holder means in that it may run along a transversal plane, i.e. a plane at right angles to the plane of the elastic holder means and may be able to be connected to the elastic holder means. The advantage of the use of a further elastic holder means lies in an improved holding function of the hearing protection means.

In one embodiment, at least one further elastic holder means may be able to be disposed in the frontal bone area or above the frontal bone area of the object under examination.

Through this arrangement a comfortable holding function may be achieved. The further elastic holder means may be disposed as a type of forehead strap and may affix the hearing protection means in a desired position.

In an exemplary embodiment, at least one supplementary elastic holder means may be able to be disposed on the at least one further elastic holder means. The at least one supplementary elastic holder means, the at least one further elastic holder means and the at least one elastic holder means may form a cap-like structure.

The supplementary elastic holder means, which can be designed as a reversible, flexible strap, may run along a sagittal plane, in the area of the head of the examination object. It is able to be connected to the further elastic holder means and the elastic holder means and, in a connected state, forms a cap-like structure. In this case the head may not be completely covered by the individual elastic straps, so that a stable fit with simultaneously possible heat emission is achieved.

In another embodiment, the length of the at least one further elastic holder means and/or the length of the at least one supplementary elastic holder means may able to be adapted.

The adaptability of the length of the at least one supplementary elastic holder means, may, for example be affixed and adjusted by the use of hook and loop strip fasteners, which may enable a good compromise between wearability comfort and the holding function.

In an alternative embodiment the at least one elastic holder means and/or the at least one further elastic holder means and/or the at least one supplementary holder means may have a width of 0.5 cm to 5.0 cm.

In the said area a good wearing comfort and at the same time a good holding function are able to be achieved.

In another embodiment the noise-protection earpieces may be able to be disposed reversibly on the at least one elastic holder means.

In particular, when the two noise-protection earpieces are able to be disposed on the elastic holder means by means of feedthrough means, they may be easily taken off and replaced by other noise-protection earpieces for example. This is especially of interest if noise-protection earpieces of different sizes are to be used for different shapes of head with the same elastic holder means.

In an exemplary embodiment, physical characteristics of the noise-protection earpieces may be able to be identified by predeterminable codes.

Physical characteristics of the noise-protection earpieces may include for example, the size or the acoustic characteristics of the noise-protection earpieces. Identifying characteristics on the basis of predeterminable codes, may include physical characteristics such as a color code, predeterminable shapes or predeterminable symbols on the noise-protection earpieces. Noise-protection earpieces with different characteristics can be distinguished by the predeterminable code and the appropriate noise-protection earpieces may be quickly selected for an individual patient.

The hearing protection means may include an acoustic tube for passing along acoustic signals to the two noise-protection earpieces.

The previously described hearing protection means may be supplemented by an acoustic tube. The acoustic tube can pass along acoustic signals of a sound source, e.g. a loudspeaker, to the two noise-protection earpieces.

Another aspect of the disclosure is a magnetic resonance tomography device with hearing protection means, wherein the magnetic resonance tomography device includes one of the previously described hearing protection means.

For example the magnetic resonance tomography device can include a sound source which creates acoustic signals which are passed on to an inventive hearing protection means with acoustic tube.

The exemplary embodiments described in greater detail below.

### BRIEF DESCRIPTION

Some of the embodiments will be described in detail, with reference to the following figures, wherein like designations denote like members, wherein:

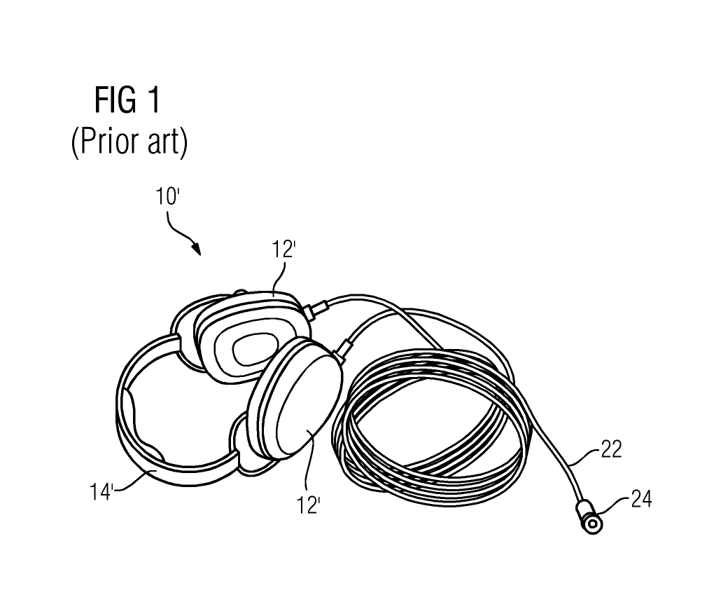

FIG. 1 shows an example of a hearing protection means in accordance with the prior art;

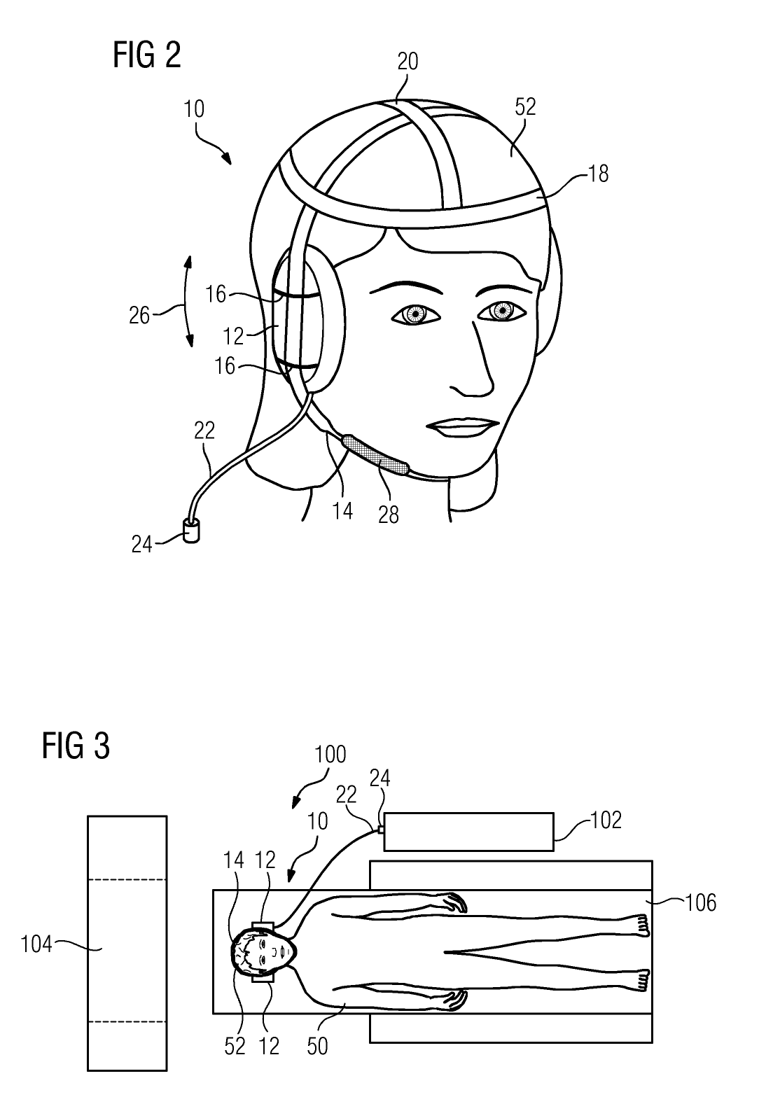

FIG. 2 shows a schematic diagram of an example of an embodiment of a hearing protection means; and

FIG. 3 shows a schematic diagram of an example of an embodiment of an inventive magnetic resonance tomography device with hearing protection means.

### DETAILED DESCRIPTION

FIG. 1 shows an example of a hearing protection means 10′ according to the prior art. The hearing protection means 10′ takes the form of headphones. It includes two noise-protection earpieces 12′ for external coverage of the outer ears, which are connected by a bracket 14′. The hearing protection means 10′ further comprises an acoustic tube 22 with an acoustic tube connection means 24 for connecting the acoustic tube 22 to a sound source.

FIG. 2 shows a schematic diagram of an example of an embodiment of a hearing protection means 10. The hearing protection means 10 may comprise two noise-protection earpieces 12 for external coverage of the two outer ears of an examination object, e.g. of a human patient. They may consist of non-magnetic materials and have a sound-deadening function, which may be achieved for example by suitable and known sound-deadening materials. An elastic holder means 14, may be a reversible, flexible strap approximately 2 cm wide, may serve to affix the noise-protection earpieces 12 around the outer ears of the examination object. The elastic holder means 14 may be in the shape of a strap and may be disposed circumferentially on the head 52 of the examination object. The elastic holder means 14 may run in a frontal plane, in the area of the lower jaw and the outer ear region. The circumferential length of the elastic holder means 14 may be able to be adapted such that a predeterminable tension is able to be set over the entire circumferential length. The degree of tension can be predetermined by a manufacturer. The elastic holder means 14 may comprise a hook and loop fastener 28. The hook and loop fastener 28 may be in the form of a hook and loop strip, for adapting the circumferential length to the individual size and shape of the head 52 and may also be used for adapting the tension. The earpieces 12 may not connected permanently to the elastic holder means 14, but may include, on the distal side of the noise-protection earpieces 12, an elastic holder means 14 guided by means of feedthroughs 16 through the noise-protection earpieces 12 so that a caudal/cranial movement 26 of the noise-protection earpieces 12 is possible. This may allow a further adaptation option to the shape and size of the head 52 of the examination object. The feedthroughs 16 may be brackets which are disposed on the outer sides of the noise-protection earpieces 12 and may form loops with the outer sides, through which the elastic holder means 14 may be guided. The use of the feedthroughs 16 allow for the two noise-protection earpieces 12 to be taken off the elastic holder means 14 and can, for example, be replaced by noise-protection earpieces of a different size. This is of particular interest if the hearing protection means 10 is to be adapted to head shapes of different sizes. The hearing protection means 10 has a further elastic holder means 18, which may be disposed circumferentially and in parallel to a transversal plane of the examination object and in the front bone area of the head 52 of the examination object. The further elastic holder means 18, which may be a reversible, flexible strap with a width of 2 cm in some embodiments, may supplement the elastic holder means 14 in such a manner that it runs on a transversal plane and is connected in this exemplary embodiment to the elastic holder means 14. The advantage of using the further elastic holder means 18 lies in an improved holding function of the hearing protection means 10. The hearing protection means 10 may further comprise a supplementary elastic holder means 20, for example, in some embodiments, a reversible, flexible strap, which is disposed on the further elastic holder means 18. The supplementary elastic holder means 20, the further elastic holder means 18 and the elastic holder means 14 may form a cap-like structure. It can be seen that the head 52 may not be completely covered by the individual elastic straps, so that a stable hold may be achieved, while at the same time emission of heat is possible. The hearing protection means 10 may also have an acoustic tube 22 with an acoustic tube connection means 24 for connection to a sound source, e.g. a loudspeaker.

FIG. 3 shows a schematic diagram of an example of an embodiment of an inventive magnetic resonance tomography device 100. The embodiment depicts a closed magnetic resonance tomography device, with a hearing protection means 10. The magnetic resonance tomography device 100 comprises a tunnel 104 into which the examination object 50, for example, a human patient, who is supported on a support device 106, such as a patient table, is to be moved. For dampening the noises created during imaging with the magnetic resonance tomography device 100, the examination object 50 wears a hearing protection means 10, which may comprise two noise-protection earpieces 12 for covering his two outer ears and an elastic holder means 14 for fixing the noise-protection earpieces 12 to the head 52 of the examination object 50. The hearing protection means 10 may also have an acoustic tube 22 with an acoustic tube connection means 24. The acoustic tube connection means 24 are connected to a processing and control device 102 of the magnetic resonance tomography device 100, through which acoustic signals, such as announcements by an operator, can be passed onto the noise protection earpieces 12.

Further embodiments and advantages of the disclosure are described in summary. Among other things, the disclosure proposes the use of a type of head cap which is made of elastic straps. In some embodiments the head may not be covered completely by the cap. Since the cap may be made of individual elastic straps it makes a stable hold possible and at the same time allow for the emission of heat. The straps disposed in the shape of a cap can be held by a chin strap on which the noise-protection earpieces can be let in with a hook and loop fastening. The hook and loop fastening is designed so that the chin strap can be adapted in size variably to the shape of the head. The noise-protection earpieces may not be permanently clamped onto the chinstrap in some embodiments. Through a small feedthrough on the distal side of the noise-protection earpieces the chin strap may be fed through such that movement upwards or downwards, i.e. in the head-foot direction, is possible. This allows a further adaptation option to the shape and size of the head of the person being examined.

For use with babies and small children, the elastic holder means, which can also be referred to as the hearing protection holder, and the noise-protection earpieces can be manufactured in different sizes, which can be distinguished for example by a predeterminable color marking. For example “green” could stand for a suitable baby size, “blue” for a suitable small child size and “red” for a child size.

Through the described features, a compact design, a flexible area of application, promoted by the possible choice of the size of hearing protection means and the possible choice of earpieces, and an improved affixation of the hearing protection earpieces, the hearing protection overall is achieved.

This type of hearing protection can likewise be transferred for use by adults. The advantage of the compact design makes it possible to use earpieces with greater sound deadening.

Among the advantages offered by the hearing protection presented are the following:
the noise-protection earpieces and the noise-protection holder are able to be adapted to the size and shape of the head;
the embodiment with elastic straps enables a good hold on the outer ear to be achieved, through which an improved sound deadening is achieved;
the small physical size of the design enables earpieces with a good sound-deadening value to be provided.

1. A hearing protection means configured for use with a magnetic resonance tomography device, comprising:
two noise-protection earpieces externally covering two outer ears of an examination object, wherein the two noise-protection earpieces are comprised of a non-magnetic material and have a sound-deadening function; and
at least one elastic holder means affixing the noise-protection earpieces around each of the two outer ears of the examination object, wherein the at least one elastic holder means has a strap-like shape able to be disposed on a head of the examination object circumferentially, running along an area of a lower jaw and a region of the outer ear, and said elastic holder means is configured to be adapted in its circumferential length such that a predeterminable tension is able to be set over the entire circumferential length.

2. The hearing protection means as claimed in claim 1, wherein the elastic holder means includes a hook and loop fastening adapting the circumferential length.

3. The hearing protection means as claimed in claim 1, wherein the two noise-protection earpieces each include a feedthrough means for longitudinal movement of the two noise-protection earpieces.

4. The hearing protection means as claimed in claim 1, wherein the hearing protection means includes a least one further elastic holder means, wherein the at least one further elastic holder means is able to be disposed circumferentially and in parallel to a transversal plane of the examination object , in the area of the head of the examination object.

5. The hearing protection means as claimed in claim 4, wherein the at least one further elastic holder means is able to be disposed in a front bone area or above the front bone area of the examination object.

6. The hearing protection means as claimed in claim 4, further comprising at least one supplementary elastic holder means configured to be disposed on the at least one further elastic holder means, wherein the at least one supplementary elastic holder means, the at least one further elastic holder means and the at least one elastic holder means form a cap-like structure.

7. The hearing protection means as claimed in claim 6, wherein the length of the at least one further elastic holder means and/or the length of the at least one supplementary elastic holder means are adaptable to the examination object.

8. The hearing protection means as claimed in claim 6, wherein the at least one elastic holder means and/or the at least one further elastic holder means and/or the at least one supplementary elastic holder means has a width of 0.5 cm to 5.0 cm.

9. The hearing protection means as claimed in claim 1, wherein the noise-protection earpieces are disposed reversibly on the at least one elastic holder means.

10. The hearing protection means as claimed in claim 9, wherein physical characteristics of the noise-protection earpieces are identifiable by a predeterminable code.

11. The hearing protection means as claimed in claim 1, further comprising an acoustic tube passing on acoustic signals to the two noise-protection earpieces.

12. A magnetic resonance tomography device comprising:
a hearing protection means having a two noise-protection earpieces comprised of a non-magnetic material externally covering outer ears of an examination object and have a sound-deadening function and at least one elastic holder means affixing the noise-protection earpieces around each of the outer ears of the examination object, wherein the at least one elastic holder means has a strap-like shape able to be disposed on a head of the examination object circumferentially, running along an area of a lower jaw and a region of the outer ear, and said elastic holder means are configured to be adapted in its circumferential length such that a predeterminable tension is able to be set over the entire circumferential length.

13. The device of claim 12, wherein the elastic holder means includes a hook and loop fastening adapting the circumferential length.

14. The device of claim 12, wherein the two noise-protection earpieces each include a feedthrough means for longitudinal movement of the two noise-protection earpieces.

15. The device of claim 12, further comprising a least one further elastic holder means, wherein the at least one further elastic holder means is able to be disposed circumferentially and in parallel to a transversal plane of the examination object , in the area of the head of the examination object.

16. The device of claim 15, wherein the at least one further elastic holder means is disposed along a front bone area or above the front bone area of the examination object.

17. The device of claim 15, further comprising at least one supplementary elastic holder means configured to be disposed on the at least one further elastic holder means, wherein the at least one supplementary elastic holder means, the at least one further elastic holder means and the at least one elastic holder means form a cap-like structure.

18. The device of claim 17, wherein the length of the at least one further elastic holder means and/or the length of the at least one supplementary elastic holder means are adaptable to the examination object.

19. The device of claim 17, wherein the at least one elastic holder means and/or the at least one further elastic holder means and/or the at least one supplementary elastic holder means has a width of 0.5 cm to 5.0 cm.

20. The device of claim 12, wherein the noise-protection earpieces are disposed reversibly on the at least one elastic holder means.
